(19) United States
(12) Patent Application Publication
(10) Pub. No.: US 2021/0174915 A1
(43) Pub. Date: Jun. 10, 2021

(54) BI-DIRECTIONAL DOCUMENTATION BUILDING SYSTEM

(71) Applicant: CERNER INNOVATION, INC., KANSAS CITY, KS (US)

(72) Inventors: Natalee Agassi, Blue Bell, PA (US); Emin Agassi, Blue Bell, PA (US)

(21) Appl. No.: 16/706,988

(22) Filed: Dec. 9, 2019

(51) Int. Cl. G16H 10/60; G06Q 50/22

(52) U.S. Cl. CPC G16H 10/60; G06Q 50/22

(57) ABSTRACT

Computerized systems and methods are provided for a dynamic custom documentation building system for building at least one custom documentation template. Upon receiving an indication to initiate the custom documentation template building process, the system receives data associated with an individual and identifies a first criteria for a custom documentation template. The system then identifies a first set of data that satisfies the first criteria and a second set of data that does not satisfy the first criteria for the custom documentation template. The second set of data is removed. The system subsequently generates the custom documentation template and provides at least a first portion of the custom documentation template to a first user on a user interface.

### BACKGROUND

Generally, when a healthcare provider is treating an individual during an encounter, the healthcare provider utilizes documentation templates in an application, such as an electronic health record, to input information regarding the individual and treatment for recordkeeping and future use. However, often times the documentation templates that a healthcare provider or other individual within a healthcare system are provided with are not contextualized and either contain too much, too little, or irrelevant fields to be completed. As such, the current documentation templates may slow down the process of effectively inputting relevant information regarding the health of the individual and opens the door for potential errors. The healthcare provider may be hindered by documentation templates that are not applicable to the current encounter situation. Further, the information input into such documentation templates may be readily available to various individuals within a healthcare system, which presents potential privacy violations. A documentation template that is contextualized and then provided in portions to different users based on their credentials may further protect the privacy of confidential health information of individuals.

### SUMMARY

This Summary is provided to introduce a selection of concepts in a simplified form that are further described below in the Detailed Description. This Summary is not intended to identify key features or essential features of the claimed subject matter, nor is it intended to be used as an aid in determining the scope of the claimed subject matter. The present invention is defined by the claims.

During an encounter with a healthcare provider, the healthcare provider documents notes regarding the encounter into an electronic health record (EHR). Often, the healthcare provider utilizes a user device, such as a laptop, during an encounter to capture relevant information related to the encounter. Applications utilized by healthcare providers include various documentation templates for inputting data. For example, many templates include fields for input of information such as a date of birth, gender, name, and medication history. Currently, documentation templates are created in one direction such that the templates are created utilizing either a bottom-up or top-bottom approach. The current documentation template building processes are not efficient. When the documentation templates are generated from the bottom-up, it means that the template builder begins with values, such as yes or no for a question, or a field associated with the question (e.g. open text box for recording responses). Then the builder builds the specific question (e.g. has the individual received a flu shot that year?), followed by the question-group, (e.g., immunizations), then template section, (e.g., immunization history), then the template name (e.g., annual adult exam, male) and template type (e.g., adult preventive care visits). This method of preparing a documentation template, while consistent, is not natural with how human thinking works. On the other hand, the top-down approach, in which specific questions or fields are first generated, followed by the answers/values, is less consistent. The mind more naturally creates the template, then each sub field/category/question, making the top-down approach feel more natural. However, a documentation template building system in which the system may operate bi-directionally or top-down and bottom-up would provide a more consistent and thorough documentation template for use users.

Additionally, current documentation templates are not contextualized. Instead, a healthcare provider may see a primary problem only, or may be provided with too much information which hinders the ability to efficiently and effectively utilize the EHR during an encounter with the individual. As such, this may lead to missing information or not accurately recording all the pertinent information.

Further, there are multiple different individuals within a healthcare system that may have access to the EHR of an individual. However, each individual does not need to access the full health record. For example, an individual working in the billing department does not need to have access to sensitive, Heath Insurance Portability and Accountability Act (HIPAA) regulated personal information such as a medical history of the individual. Instead, the billing individual needs to only see pertinent billable components of the treatment procedures and medications provided in order to determine the appropriate billing codes based on insurance. Likewise, a healthcare provider is not interested in viewing the billing or insurance information for the patient and does not need to see that information during the patient encounter. Therefore, there is a need for a custom documentation template building process that would customize the documentation template based on a user's input, among other things. Additionally, there is a need for a system that would allow different individuals within a healthcare system and care team to view different portions and interpretations of the template that are relevant to their individual roles.

Embodiments of the present invention generally relate to computerized systems and methods for building at least one custom documentation template for presentation to and input by at least one user. The bi-directional documentation building system utilizes indications received from a first user, data associated with an individual from a first source, and determinations of a first criteria for a custom documentation template to generate the custom documentation template. The system identifies a first set of data that satisfies the first criteria determined and a second set of data that does not satisfy the first criteria determined. The second set of data that does not satisfy the first criteria is removed and then the custom documentation template is generated with the first set of data. The system then provides at least a first portion of the custom documentation template to the first user on a user interface.

### BRIEF DESCRIPTION OF THE SEVERAL VIEWS OF THE DRAWINGS

The present invention is described in detail below with reference to the attached drawing figures, wherein:

FIG. 1 is a block diagram of an exemplary system architecture in which embodiments of the invention may be employed, in accordance with aspects herein;

FIG. 2 is an exemplary system architecture suitable to implement embodiments of the present invention, in accordance with aspects herein;

FIGS. 3a-3b illustrate exemplary custom documentation templates, in accordance with aspects herein;

FIG. 4 illustrates an exemplary process of the generating a custom documentation template by a dynamic bi-directional documentation building system, in accordance with aspects herein;

FIGS. 5a-5c illustrate different portions of the custom documentation template described in FIG. 4 that are provided to different users, in accordance with aspects herein;

FIG. 6 illustrates an exemplary method for building at least one custom documentation template, in accordance with aspects herein;

FIG. 7 illustrates another exemplary method for building at least one custom documentation template, in accordance with aspects herein;

FIG. 8 illustrates a base custom documentation template and a custom documentation template generated based the base custom documentation template, in accordance with aspects herein;

FIG. 9 illustrates another base custom documentation template and a custom documentation template generated based the base custom documentation template, in accordance with aspects herein; and

FIG. 10 illustrates exemplary template segments for generating a custom documentation template, in accordance with aspects herein.

### DETAILED DESCRIPTION

The subject matter of the present invention is described with specificity herein to meet statutory requirements. However, the description itself is not intended to limit the scope of this patent. Rather, the inventors have contemplated that the claimed subject matter might also be embodied in other ways, to include different steps or combinations of steps similar to the ones described in this document, in conjunction with other present or future technologies. Moreover, although the terms “step” and/or “block” may be used herein to connote different components of methods employed, the terms should not be interpreted as implying any particular order among or between various steps herein disclosed unless and except when the order of individual steps is explicitly described.

The effective management of healthcare documentation systems, including electronic health records, is complex and requires the input of multiple individuals with various roles. Generally, the process begins with an individual arriving at a clinic, clinician's office, emergency department, or any other healthcare facility with a healthcare concern. When the individual enters, for example the emergency department, they first check in and provide basic personal information such as their name, date of birth, insurance information, and indicate the healthcare concern for which they are seeking treatment. After this, the individual is brought to triage where a hospital employee (e.g. nurse) takes the individual's vital signs, additional medical history, and additional personal information, which is all entered into the individual's electronic health record/account. However, due to the complexity of the overall system, sometimes, the hospital employee or healthcare provider is provided with electronic documentation templates that are either too broad, too narrow, or may not be appropriately tailored for inputting the necessary information for effective healthcare management. As such, this often leads to inefficient documentation templates that do not capture the information needed appropriately. Additionally, when considering HIPAA and other privacy regulations, such templates present potential issues. For example, documentation templates that are accessible to all and not contextualized based on the individual user, allow for unnecessary and sometimes confidential information to be released to multiple users who do not possess the requirements for viewing such information.

Often times, documentation templates are created within a healthcare management system for managing the input of data from multiple different users. As such, the documentation templates are often not customized for each individual user. Therefore, when a healthcare provider initiates an encounter with an individual, the healthcare provider may be presented with multiple fields to input data. As used herein, encounter generally refers to any interaction between an individual and a clinician that results in one or both of an access of the individual's EHR or documentation within the individual's EHR. Depending on the individual and nature of the encounter, the documentation template may have too few, too many, or more fields than are fully relevant. For example, if an individual is being seen by a healthcare provider in the emergency room, the documentation template presented for inputting encounter data regarding the encounter and individual is sometimes too cumbersome due to the lack of customization. Encounter data, as used herein, refers generally to any data that is populated or documented in a customized template. As such, non-customized and generic documentation templates may lead to ineffective input of data by the healthcare provider since the healthcare provider may be presented with a documentation template that is not relevant to the current encounter. Therefore, the healthcare provider's analysis may be hindered due to the need to review and input irrelevant data for the individual into the documentation template. In other aspects, the healthcare provider may overlook inputting key data due to the generic nature of the documentation template, which thereby may lead to treatment or recordkeeping errors.

In light of these issues, a bi-directional documentation building system for building at least one custom documentation template provides a solution to the above limitations of current documentation templates. Historically, the documentation templates were generated from the bottom up, so that each value would first be determined (e.g. each answer—yes or no), followed by a determination of each associated question (e.g. is the individual a smoker?). The current process does not naturally align with human thinking as an individual, such as a healthcare provider, does not first provide an answer to a question prior to asking the question. A system that is bi-directional allows for creation of custom documentation templates in two directions, from top-down and from bottom-up. The bi-directional nature of the system allows for the system to more effectively capture the values and parameters, such as the types of data, to be included into the custom documentation template. Additionally, a system that is able to contextualize the custom documentation template based on input received from a user, data associated with an individual, and determination of the criteria for the documentation template provides a more user-friendly, efficient, accurate, and improved documentation template for use in a healthcare management system. It also allows for the creation of custom documentation templates comprising multiple portions, some or all of which, may be provided to a user for input via a user interface. The custom nature and contextualization additionally enhances the protection of confidential health information by allowing the system to provide only the necessary portion of a customized template to the user for review and input. This prevents unnecessary and unlawful dissemination of private health information (e.g. an individual in the accounting department accessing and reviewing the full medical record of an individual) to unauthorized individuals.

As described in further detail below, the custom documentation building system for building at least one custom documentation template for presentation to a user is a bi-directional system that is contextualized and generates a custom documentation template for use by an individual user. The custom documentation building system streamlines the layouts and styles of custom documentation templates generated, supports multiple configurable views, and filters document content based on user role, clinical setting, document type and other variables. In aspects, the dynamic custom documentation building system receives one or more indications to initiate a custom documentation template building process from a first user. The system receives data associated with an individual from at least a first source and determines a first criteria for a custom documentation template based on the data associated with the individual from the first source. The system identifies a first set of data that satisfies the first criteria for the custom documentation template and identifies a second set of data that does not satisfy the first criteria for the custom documentation template. The second set of data that does not satisfy the first criteria for the custom documentation template is removed. A custom documentation template comprising the first set of data that satisfies the first criteria for the custom documentation template is generated. At least a first portion of the custom documentation template is provided to a first user on a user interface.

In other aspects, the dynamic bi-directional documentation building system for building at least one custom documentation template for presentation to and input by at least one user comprises one or more processors that are configured to receive one or more indications to initiate a custom documentation template documentation building process from a first user for a first encounter associated with an individual. The system also receives one or more template segments associated with the first encounter from a first source. The system determines a first criteria for a custom documentation template based on the one or more template segments associated with the first encounter from the first source. The system identifies a first set of template segments that satisfy the first criteria for a custom documentation template and a second set of template segments that do not satisfy the first criteria for the custom documentation template. The system receives data associated with the individual from a second source based on the determined first criteria for the custom documentation template. The custom documentation template is generated comprising the first set of template segments that satisfy the first criteria for the custom documentation template and the data associated with the individual. Following this, at least a portion of the custom documentation template is provided to the first user on a user interface.

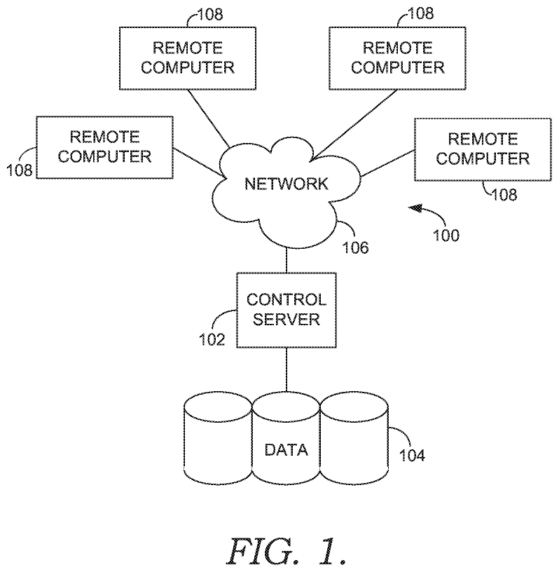

Beginning with FIG. 1, an exemplary computing environment suitable for use in implementing embodiments of the present invention is shown. FIG. 1 is an exemplary computing environment (e.g., health-information computing-system environment) with which embodiments of the present invention may be implemented. The computing environment is illustrated and designated generally as reference numeral 100. The computing environment 100 is merely an example of one suitable computing environment and is not intended to suggest any limitation as to the scope of use or functionality of the invention. Neither should the computing environment 100 be interpreted as having any dependency or requirement relating to any single component or combination of components illustrated therein. It will be appreciated by those having ordinary skill in the art that the connections illustrated in FIG. 1 are also exemplary as other methods, hardware, software, and devices for establishing a communications link between the components, devices, systems, and entities, as shown in FIG. 1, may be utilized in the implementation of the present invention. Although the connections are depicted using one or more solid lines, it will be understood by those having ordinary skill in the art that the exemplary connections of FIG. 1 may be hardwired or wireless, and may use intermediary components that have been omitted or not included in FIG. 1 for simplicity's sake. As such, the absence of components from FIG. 1 should not be interpreted as limiting the present invention to exclude additional components and combination(s) of components. Moreover, though devices and components are represented in FIG. 1 as singular devices and components, it will be appreciated that some embodiments may include a plurality of the devices and components such that FIG. 1 should not be considered as limiting the number of a device or component.

The present technology might be operational with numerous other special-purpose computing system environments or configurations. Examples of well-known computing systems, environments, and/or configurations that might be suitable for use with the present invention include personal computers, server computers, hand-held or laptop devices, multiprocessor systems, microprocessor-based systems, set top boxes, programmable consumer electronics, network PCs, minicomputers, mainframe computers, distributed computing environments that include any of the above-mentioned systems or devices, and the like.

The present invention may be operational and/or implemented across computing system environments such as a distributed or wireless “cloud” system. Cloud-based computing systems include a model of networked enterprise storage where data is stored in virtualized storage pools. The cloud-based networked enterprise storage may be public, private, or hosted by a third party, in embodiments. In some embodiments, computer programs or software (e.g., applications) are stored in the cloud and executed in the cloud. Generally, computing devices may access the cloud over a wireless network and any information stored in the cloud or computer programs run from the cloud. Accordingly, a cloud-based computing system may be distributed across multiple physical locations.

The present technology might be described in the context of computer-executable instructions, such as program modules, being executed by a computer. Exemplary program modules comprise routines, programs, objects, components, and data structures that perform particular tasks or implement particular abstract data types. The present invention might be practiced in distributed computing environments where tasks are performed by remote processing devices that are linked through a communications network. In a distributed computing environment, program modules might be located in association with local and/or remote computer storage media (e.g., memory storage devices).

With continued reference to FIG. 1, the computing environment 100 comprises a computing device in the form of a control server 102. Exemplary components of the control server 102 comprise a processing unit, internal system memory, and a suitable system bus for coupling various system components, including data store 104, with the control server 102. The system bus might be any of several types of bus structures, including a memory bus or memory controller, a peripheral bus, and a local bus, using any of a variety of bus architectures. Exemplary architectures comprise Industry Standard Architecture (ISA) bus, Micro Channel Architecture (MCA) bus, Enhanced ISA (EISA) bus, Video Electronic Standards Association (VESA) local bus, and Peripheral Component Interconnect (PCI) bus, also known as Mezzanine bus.

The control server 102 typically includes therein, or has access to, a variety of non-transitory computer-readable media. Computer-readable media can be any available media that might be accessed by control server 102, and includes volatile and nonvolatile media, as well as, removable and nonremovable media. By way of example, and not limitation, computer-readable media may comprise computer storage media and communication media. Computer storage media includes volatile and nonvolatile, removable and non-removable media implemented in any method or technology for storage of information such as computer-readable instructions, data structures, program modules or other data. Computer storage media includes, but is not limited to, RAM, ROM, EEPROM, flash memory or other memory technology, CD-ROM, digital versatile disks (DVD) or other optical disk storage, magnetic cassettes, magnetic tape, magnetic disk storage or other magnetic storage devices, or any other medium which can be used to store the desired information and which can be accessed by control server 102. Computer-readable media does not include signals per se.

Communication media typically embodies computer-readable instructions, data structures, program modules or other data in a modulated data signal such as a carrier wave or other transport mechanism and includes any information delivery media. The term “modulated data signal” means a signal that has one or more of its characteristics set or changed in such a manner as to encode information in the signal. By way of example, and not limitation, communication media includes wired media such as a wired network or direct-wired connection, and wireless media such as acoustic, RF, infrared and other wireless media. Combinations of any of the above should also be included within the scope of computer-readable media.

The control server 102 might operate in a computer network 106 using logical connections to one or more remote computers 108. Remote computers 108 might be located at a variety of locations including operating systems, device drivers and the like. The remote computers might also be physically located in traditional and nontraditional clinical environments so that the entire medical community might be capable of integration on the network. The remote computers might be personal computers, servers, routers, network PCs, peer devices, other common network nodes, or the like and might comprise some or all of the elements described above in relation to the control server. The devices can be personal digital assistants or other like devices. Further, remote computers may be located in a variety of locations including in a medical or research environment, including clinical laboratories (e.g., molecular diagnostic laboratories), hospitals and other individual settings, veterinary environments, ambulatory settings, medical billing and financial offices, hospital administration settings, home medical environments, and clinicians' offices. Medical providers may comprise a treating physician or physicians; specialists such as surgeons, radiologists, cardiologists, and oncologists; emergency medical technicians; physicians' assistants; nurse practitioners; nurses; nurses' aides; pharmacists; dieticians; microbiologists; laboratory experts; laboratory technologists; genetic counselors; researchers; veterinarians; students; and the like. The remote computers 108 might also be physically located in nontraditional clinical environments so that the entire medical community might be capable of integration on the network. The remote computers 108 might be personal computers, servers, routers, network PCs, peer devices, other common network nodes, or the like and might comprise some or all of the elements described above in relation to the control server 102. The devices can be personal digital assistants or other like devices.

Computer networks 106 comprise local area networks (LANs) and/or wide area networks (WANs). Such networking environments are commonplace in offices, enterprise-wide computer networks, intranets, and the Internet. When utilized in a WAN networking environment, the control server 102 might comprise a modem or other means for establishing communications over the WAN, such as the Internet. In a networking environment, program modules or portions thereof might be stored in association with the control server 102, the data store 104, or any of the remote computers 108. For example, various application programs may reside on the memory associated with any one or more of the remote computers 108. It will be appreciated by those of ordinary skill in the art that the network connections shown are exemplary and other means of establishing a communications link between the computers (e.g., control server 102 and remote computers 108) might be utilized.

In operation, an organization might enter commands and information into the control server 102 or convey the commands and information to the control server 102 via one or more of the remote computers 108 through input devices, such as a keyboard, a microphone (e.g., voice inputs), a touch screen, a pointing device (commonly referred to as a mouse), a trackball, or a touch pad. Other input devices comprise satellite dishes, scanners, or the like. Commands and information might also be sent directly from a remote medical device to the control server 102. In addition to a monitor, the control server 102 and/or remote computers 108 might comprise other peripheral output devices, such as speakers and a printer.

Although many other internal components of the control server 102 and the remote computers 108 are not shown, such components and their interconnection are well known. Accordingly, additional details concerning the internal construction of the control server 102 and the remote computers 108 are not further disclosed herein.

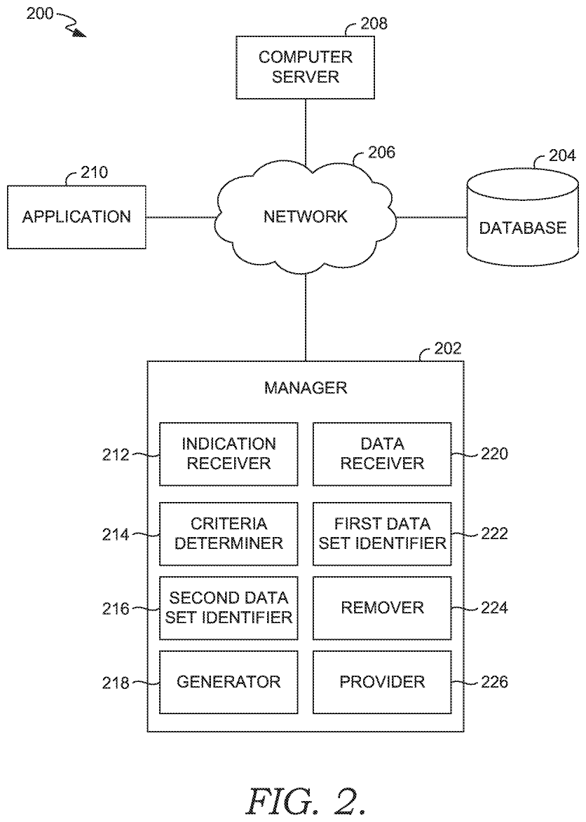

Turning now to FIG. 2, a dynamic bi-directional documentation building system 200 for building at least one custom documentation template for presentation and input by at least one user is depicted. The exemplary dynamic bi-directional documentation building system 200 is merely an example of one suitable system and is not intended to suggest any limitation as to the scope of use or functionality of embodiments of the present invention. Neither should the dynamic bi-directional documentation building system 200 be interpreted as having any dependency or requirement related to any single component or combination of components illustrated herein.

The exemplary dynamic bi-directional documentation building system 200 comprises a manager 202, a database, 204, a network 206, a computer server 208, and an application 210. As shown, FIG. 2 includes one manager 202, one database 204, one network 208, and one application 210. However, it is contemplated that the dynamic bi-directional documentation building system 200 may comprise more than one of each of these components depending on the needs of the dynamic bi-directional documentation building system 200. For example, the dynamic bi-directional documentation building system 200 may comprise more than one database 204, which may be located remotely or on the cloud.

As depicted, the dynamic bi-directional documentation building system 200 comprises a manager 202. It will be appreciated that some or all of the subcomponents of the manager 202 may be accessed via the network 206 and may reside on one or more devices. Additionally, the manager 202 may also be integrated into the application 210. Further, in some embodiments, one or more of the illustrated components may be implemented as a stand-alone application. The components described are exemplary in nature and in number and should not be construed as limiting. Any number of components may be employed to achieve the desired functionality within the scope of the embodiments hereof.

Generally, the manager 202 is configured to build at least one custom documentation template for presentation to a first user. The Manager 202 is the orchestrator of the custom documentation template generation process. It sets up the process and invokes each sub component in the generation of the custom documentation template. Upon receiving the custom documentation template, the first user may record information related to an encounter with an individual into the custom documentation template. As shown, the manager 202 is comprised of several components including: an indication receiver 212, a data receiver 220, a criteria determiner 214, a first data set identifier 222, a second data set identifier 216, a remover 224, a generator 218 and a provider 226. In embodiments, the manager 202 can include any number of components necessary for building at least one custom documentation template.

The indication receiver 212 within manager 202 is configured to receive one or more indications to initiate a custom documentation template building process from a first user. The one or more indications to initiate the custom documentation template building process are indications that instruct the indication receiver 212 to initiate the documentation template building process. Such indications may occur when the first user, such as a healthcare provider, inputs certain information into an application (such as application 210) or electronic health record. Exemplary one or more indications may include input of biographical information, such as the name, date of birth, or social security number of an individual. Additionally, one or more indications may also include details regarding the encounter taking place with the healthcare provider. For example, the one or more indications may be a cardiologist initiating care related to managing the cardiac care of a patient due to certain conditions such as an arrhythmia. In this instance, the cardiologist may input certain relevant information regarding the patient's symptoms or heart condition (e.g. electrocardiogram (E.K.G.) values) that will be the indications sent to the system to initiate a custom documentation template building process for the cardiologist that includes criteria relevant to the individual and their cardiac care. In other aspects, the one or more indications received may be from a variety of other sources, such as a billing clerk or an administrative assistant preparing materials for a healthcare providers' upcoming scheduled encounters with various individuals. As such, the one or more indications that the indication receiver 212 receives are not limited and may change depending on the specific situation.

Once the dynamic bi-directional documentation building system 200 has received the one or more indications to initiate the custom documentation template building process from the first user, a data receiver 220 will receive data associated with an individual from a first source. The data received by the data receiver 220 may include, but is not limited to, biographical data such as the name, date of birth, or gender. The data receiver 220 may also receive data such as previous medical history, current or past medications, or family history form the first source. In some aspects, the first source may be a database, such as database 204. In other aspects, the first source may be an application within dynamic bi-directional documentation building system 200, such as application 210. It is contemplated that the application 210 may be an electronic health record system that stores profiles for multiple individuals. The data receiver 220 may receive data from the electronic health record related to the individual. In some circumstances, the data receiver 220 may receive all the data available regarding the individual. In other circumstances, the data receiver 220 may receive limited data based on the one or more indications received by the indication receiver 212. For example, the data received by the data receiver 220 may be limited to data associated with the individual for a certain determined time period (e.g. last 5 years). In other aspects, the data received by the data receiver 220 may be limited to data relevant to a specific encounter taking place or individual healthcare provider (e.g. data receiver 220 may receive only gynecological data for an individual from the EHR for a visit with the gynecologist). Additionally, both the indication receiver 212 and the data receiver 220 are envisioned to utilize in-memory high performance caches such as Redis™ or Hazelcast™, which are in-memory databases populated from different data sources such as an external database or application programming interfaces (API). These make it possible to retrieve external data required for the custom documentation template building process.

Once the data associated with the individual is received by the data receiver 220, a criteria determiner 214 determines a first criteria for a custom documentation template based on the data associated with the individual from the first source. Criteria determiner 214 filters the data criteria received by the indication receiver 212 and the data receiver 220. The determination of the first criteria occurs quickly since it is powered by the in-memory caches. For example, if the one or more indications received by the indication receiver 212 are the date of birth, name, and gender of the individual and the data received by the data receiver 220 for the individual is data related to the individuals cardiac health, the criteria determiner 214 may determine that the first set of criteria for the custom documentation template should include all questions and associated values or fields that relate to cardiac health. In other aspects, the criteria determiner may determine that the first criteria is data within an electronic health record associated with the individual. The first criteria may further include specialty driven ranges for certain values (e.g. minimum to maximum oxygen level range) and age driven rules (e.g. only certain criteria is applicable for an individual in the age range of 18-30 versus 55-70 years old). The first criteria determined by the criteria determiner 214 may vary and any and all variations are contemplated herein.

Once the first criteria has been identified, a first data set identifier 222 will identify a first set of data, from the data associated with the individual received by the data receiver 220, that satisfies the first criteria for the custom documentation template. Continuing with the cardiac example, the first set of data identified by the first data set identifier 222 may include, but is not limited to, heart rate, blood pressure, EKG values, stress test values, medications, heart rhythm, and the like for the individual. Additionally, the first set of data that satisfies the first criteria for the custom documentation template may be a single piece of data, such as a stress test value. Based on the received data associated with the individual from at least a first source and the first criteria determination, the first data set identifier will identify at least one piece of data that meets the first criteria for the custom documentation template.

Next, the second data set identifier 216 will identify a second set of data, from the data associated with the individual received by the data receiver 220, that does not satisfy the first criteria for the custom documentation template. Using the example above, the second set of data that is determined not to satisfy the first criteria may be data received for the individual from the first source that is not pertinent to any cardiac care. For example, this might include data related to a prior appendectomy surgery or gynecological history, which may have no impact on the current cardiac care. Depending on the first criteria determined, the second set of data that does not satisfy the first criteria will vary. In some aspects, the second set of data may be a broad set of data, such as all data associated with the individual that is not relevant to the heart or any cardiac care. In other aspects, the second set of data may be more limited and include data associated with the individual related to billing and insurance.

Once the second set of data has been identified, the remover 224 removes the second set of data that does not satisfy the first criteria for the first custom documentation template from the data received by the data receiver 220 associated with the individual. The remover 224 removes the second set of data so that only the first set of data that satisfies the first criteria for the custom documentation template remains for the generation of the custom documentation template. This contextualization by the dynamic bi-directional documentation building system 200 prevents unnecessary data fields, such as billing information, to be included in the custom documentation template for the user. The first data set identifier 222, the second data set identifier 216, and the remover 224 dynamically determine the context and specific data fields to be included in the custom documentation templates.

Upon removal of the second set of data, a generator 218, generates a custom documentation template that comprises the first set of data that satisfies the first criteria for the custom documentation template. The custom documentation template is contextualized based on the data associated with the first individual and the first criteria determination. In other words, the generator 218 collects the fields determined for inclusion in the custom documentation template based on the first criteria and second criteria determinations. For example, if the first criteria for the custom documentation template was data related to cardiac care for the individual, the custom documentation template would include data fields relating to cardiac care. In some aspects, some of the portions of data would already be complete based on the data associated with the individual received from the first source. As such, data fields within the custom documentation template such as a date of birth, gender, social security number, previous medical history, if available, may already be completed within the custom documentation template. Other fields, such as symptoms, blood pressure, electrocardiogram results, and treatment provided may remain blank for input by the first user based on the individual encounter. Additionally, while the exemplary custom documentation template may include data fields related to cardiac care, this may include fields that relate to billing for the care provided by the healthcare provider. For example, the custom documentation template may include fields for inputting in the billing codes for insurance associated with the treatment provided and other relevant insurance and financial information.

The dynamic bi-directional documentation building system 200 may generate the custom documentation template so that more than one user may utilize the custom documentation template created. As such, when the custom documentation template is generated by the generator 218, the provider 226 may provide at least a first portion of the custom documentation template to the first user on a user interface. Therefore, if the custom documentation template building process was initiated by a cardiologist prior to an encounter with the specific individual, the provider 226 may provide a first portion (relevant to the cardiologist's role) of the custom documentation template to the cardiologist for use prior to, during, or after the encounter with the individual for input regarding cardiac care of the individual.

In some aspects, the provider may provide the custom documentation template to the user in its entirety (i.e., no portions are removed). In other aspects, only certain portions of the custom documentation template may be provided by the provider 226. For example, the custom documentation template may comprise data related to the care of the individual within a specific healthcare system. If the individual is being seen by a cardiologist within a hospital setting, the custom documentation template may comprise data fields related to care of the individual while at the hospital. As such, this will include fields related to billing, triage at the emergency room, and prior visits to the hospital or data associated with the individual relating to encounters with other healthcare providers that may be associated with that healthcare system. In this case, the first criteria identified may be limited to a time frame (e.g. last year, last 90 days, etc.) related to the specific individual. When the custom documentation template is generated, there may be numerous fields of data that may already be completed (e.g. information related to previous care, biographical information for the individual such as date of birth, gender, etc.) and several fields for input relating to the present encounter, treatment, medications, and billing. As such, while all of these fields may be included within the first set of data that satisfies the first criteria for the custom documentation template, all of the data fields may not be relevant for the first user (e.g. cardiologist). As such, the provider 226 may provide at least a first portion of the custom documentation template to the first user for input. Other portions of the custom documentation template, such as portions related to billing, may be provided to a second user.

Additionally, in aspects, the portion of the custom documentation template provided by the provider 226 may depend on the identity of the first user and credentials of the first user. Due to HIPAA and other privacy related laws and regulations, it is critical to maintain the security and privacy of each individuals' confidential and sensitive healthcare information. As such, the dynamic bi-directional documentation building system 200 may further verify one or more credentials related to the first user prior to when the provider 226 provides the at least first portion of the custom documentation template to the first user. Additionally, the dynamic bi-directional documentation building system 200 may verify one or more credentials for additional users prior to providing a portion of the custom documentation template to those users.

In other aspects, the dynamic bi-directional documentation building system for building at least one custom documentation template for presentation and input by at least one user may include first receiving template segments or portions or fields for completion by a user (e.g. treatment provided, medication history, etc.) and removing template segments that do not meet the first criteria. In this aspect, the system would still begin by receiving one or more indications to initiate a custom documentation template documentation building process from a first user for a first event associated with an individual. In other words, the indication receiver 212 may still receive an indication from a first user (e.g. a clinician) to initiate a custom documentation building process for an individual for a specific event, such as an emergency room visit. The system may receive one or more template segments associated with the first event from a first source. In this aspect, the dynamic bi-directional documentation system 200's manager 202 may comprise additional components such as a template segment receiver (not shown). In yet other aspects, other components of the manager 202 shown in FIG. 2, such as the data receiver 220 or indication receiver 212 may receive the template segments from a first source. The first source may be the database 204 which stores various template segments created previously by the dynamic bi-directional documentation building system 200 based on known needs of the healthcare management system or may be a separate application that has template segments stored. Additionally, in aspects, when the first source is an application, the application may comprise a set of rules and data fields to be included in the custom documentation template.

Once the one or more template segments are received, the criteria determiner 214 will determine a first criteria for the custom documentation template based on the one or more template segments associated with the first event from the first source. The manager 202 of dynamic bi-directional documentation building system 200, via a component such as, but not limited to, the first data set identifier 222 or a first template segment set identifier (not shown), may identify a first set of template segments that satisfy the first criteria identified. Additionally, the manager 202, via either the same component or another component, such as the second data set identifier 216 or a second template segment set identifier (not shown), may identify a second set of template segments that do not satisfy the first criteria for the custom documentation template.

Once the first set of template segments and second set of template segments are identified, the remover 224 may remove the second set of templates that do not satisfy the first criteria. The data receiver 220 may receive data associated with the individual from a second source based on the determination of the first criteria for the custom documentation template. For example, the data receiver 220 may receive data related to the individual from an electronic health record, an application such as application 210, a database 204, or any other source that the data receiver 220 is able to communicate with via the network 206.

The generator 218 may generate a custom documentation template comprising the first set of template segments that satisfy the first criteria for the custom documentation template and the data associated with the individual. Generating a custom documentation template in this manner will allow for additional customization of the documentation template based on the individual, the event, and indications received by the first user. Additionally, this process allows the dynamic bi-directional documentation building system 200 to provide, in the custom documentation template, the data already available regarding the individual. This will allow for a more complete and effective documentation template since the first user will receive the documentation template with certain information already complete based on the data available for the individual, thereby saving the healthcare provider time in entering (or re-entering) such information and potentially preventing the possibility of irregularities or errors due to multiple user inputs. Once the custom documentation template is generated, the provider 226 will provide at least a first portion of the custom documentation template to the first user on a user interface. The portion of the custom documentation template provided, as discussed herein, may be based on the user's credentials. Further, it is contemplated that, in some aspects, the provider 226 may further provide a template preview to the user prior to the generation of the final custom documentation template.

As mentioned, in aspects, the provider 226 may provide different portions or different customized documentation templates to different users based on the individual user's need. Customized templates may be provided for several individuals connected with the specific event taking place. In such instances, the customized documentation templates generated may be provided not only to healthcare providers, but also to caregivers, patients, and individuals associated with billing or accounting for the event.

In yet other aspects, the dynamic bi-directional documentation building system for building at least one custom documentation template for presentation and input by at least one user may include beginning with one or more base custom documentation template types. The base custom documentation template may be categorized by the specialty of the healthcare provider. For example, if the event taking places occurs at a cardiologist's office, there may be a set number of base custom documentation templates to begin the process of preparing the customized documentation template form based on the types of medical information collected for a cardiologist visit. A base custom documentation template generated for a cardiologist may include questions related specifically to cardiac care such as heart rate, blood pressure, and oxygen level. By contrast, a base custom documentation template for a gastroenterologist visit may have questions such as date of last colonoscopy and number of bowel movements per day. Customizing the base custom documentation template by specialty, rather than by other categories, such as provider name, are more effective as providers within a healthcare practice or healthcare system may leave. Therefore, categorizing the base template by specialty such as cardiology, infectious disease, or others, allows the information input by such users to be readily available and locatable as needed. Additionally, at the time of the encounter, when the base custom documentation template type is selected (e.g., emergency department provider note), the contextualized questions and associated values can be retrieved and presented on the user interface. For example, if the patient is female, questions related to the last menstrual period will become relevant to populate in the examination section of the custom documentation template for user input.

Additionally, the dynamic bi-directional documentation building system may comprise a library of questions that are populated into the base custom documentation template type (e.g. Heart rate, EKG, medications, cholesterol levels) and then a library of allowable value sets for each of the questions (e.g. heart rate between 0-200 beats per minute). Based on the one or more base custom documentation template types, library of questions, and library of allowable value sets, the bi-directional documentation building system may create multiple versions of a custom documentation template for the type event (e.g. cardiologist appointment) that further comprise different versions and sections based on the data received by the data receiver 220.

In further aspects, base custom documentation templates may be categorized by template type and may be more generic and not specific to the type of healthcare provider. For example, the base custom documentation template may be categorized as a progress note, a discharge summary, admission note, or a wellness exam. These types of base templates may remain general so that they can be used by any type of healthcare provider or may be further customized. For example, a generic progress note for cardiologist and a gastroenterologist may have the same questions such as age, sex, weight, and current medications list and as such, the base custom documentation template type for these two healthcare provider specialties may be the same or very similar. In other aspects, it is contemplated that a base documentation template for a progress note may be distinctly different from one type of provider or encounter to another.

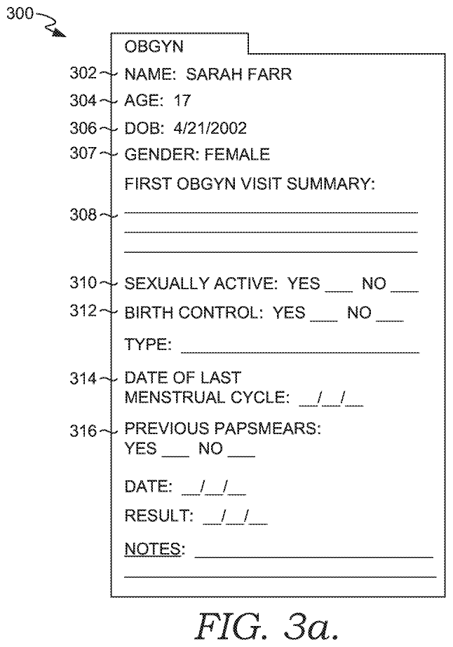

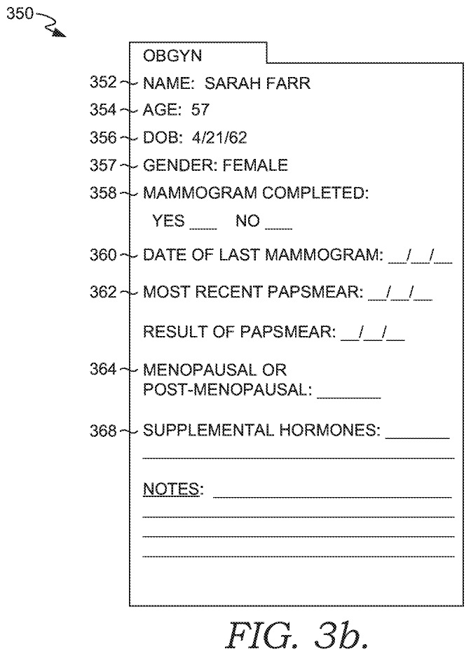

Returning to the figures, FIGS. 3a-3b illustrate two exemplary custom documentation templates. The documentation templates are related to a gynecological encounter for an individual, Sarah Farr, and are generated by the generator 218 based on the first criteria determination by the criteria determiner 214. Based on the one or more indications to initiate a custom documentation template received by the indication receiver 212, the data received by the data receiver 220 associated with the individual, and the first criteria determination by the criteria determiner 214, two different custom documentation templates are generated.

FIG. 3a illustrates a custom documentation template 300. In custom documentation template 300, various data fields are included such as name 302, age 304, date of birth (DOB) 306, gender 307, first OBGYN visit summary 308, sexually active 310, birth control 312, date of last menstrual cycle 314, and previous pap smear 316. This custom documentation template is contextualized and based on the indication the dynamic bi-directional documentation building system 200 received from the first user. As such, when the dynamic bi-directional documentation building system 200 received the indication to initiate the custom documentation template documentation building process for individual Sarah Farr for an encounter with the gynecologist, the dynamic bi-directional documentation building system 200 may generate custom documentation template 300 for Sarah Farr for the first user (e.g., gynecologist) to complete during the encounter with individual Sarah Fan. Based on the data received for Sarah Farr (e.g., date of birth, age, gender, etc.) from a first source (e.g., an electronic health record), the criteria determiner 214 may determine that the first criteria for the custom documentation template includes those data fields that are applicable to a female, age 17. As such, the criteria determiner 214 outlines the first criteria for the custom documentation template for individual Sarah Farr, a 17-year old female. Then the first data set identifier 222 may identify a first set of data that satisfied the first criteria for the custom documentation template, which includes data related to gynecological care specific to a female in a certain age range. As such, the first set of data identified by the first data set identifier 222 includes data fields such as birth control 312 and date of last menstrual cycle 314. Additionally, the second data set identifier 216 may identify a second set of data that did not satisfy the first criteria and was therefore removed by remover 224. In this case, such data may have included data fields such as those seen in FIG. 3b, including, but not limited to, date of last mammogram 360. Based on the identification of the first set of data that met the first criteria for the custom documentation template desired in this example for the gynecological encounter, the generator 218 may generate custom documentation template 300 and the provider 226 may provide at least a first portion of custom documentation template 300 illustrated in FIG. 3a.

By contrast, FIG. 3b illustrates how the dynamic bi-directional documentation building system 200 contextualizes different indications received from the first user, the data associated with the individual from a first source, and the determined first criteria to generate another exemplary custom documentation template 350. As shown in FIG. 3b, when individual Sarah Farr's date of birth is changed and the resulting age increases from 17 to 52, the applicable first set of data for the custom documentation template changes. The system dynamically determines that the first criteria for Sarah Fan, a 57-year old female, should include data related to a post-menopausal female. As such, in this scenario, the criteria determiner 214 determines that the first criteria for the custom documentation template should include that data which is relevant to a gynecological encounter with a 57-year old female. Therefore, the first set of data identified by the first data set identifier 222 includes fields for completion by the first user including whether a mammogram has been completed at 358, the date of the last mammogram 360, most recent pap smear 362, whether the individual is menopausal or post-menopausal 364, and whether the individual is taking any supplemental hormones 368. This first set of data satisfies the first criteria as it is applicable to the gynecological care of a 57-year old woman.

The second set of data identified by the second data set identifier 216 and removed by the remover 224 may include data found in FIG. 3a that is applicable to a 17-year old female. For example, the second set of data may include the date of last menstrual cycle 314 or whether the individual is on birth control 312. While a documentation template for a first user to input data could include the information seen in both FIGS. 3a and 3b, the contextualization provided by the dynamic bi-directional documentation building system 200 in order to customize the custom documentation template 300 and 350 based on the specific individual and encounter make the custom documentation template more usable for a healthcare provider. It allows the healthcare provider to quickly review and identify the relevant medical information related to the individual for the specific encounter and input/document relevant information for treatment and recordkeeping. By contextualizing and presenting a customized documentation template as described herein, the first user can quickly review, digest, and document the relevant information for an encounter.

In other aspects, the custom generated documentation template for the gynecological visit example discussed above may actually include all of the data seen in FIGS. 3a and 3b. For example, if Sarah Farr had been seeing the same gynecologist from age 17 to age 57, the data associated with Sarah Farr in the electronic health record may include all information seen in FIGS. 3a and 3b. If the gynecologist is a part of a larger healthcare system, the first source may also have data associated with Sarah Farr related to care from other healthcare providers (e.g. endocrinologist, cardiologist, etc.). As such, in this case, the first set of data that satisfies the first criteria may be all the data that is related to the gynecological history and care of Sarah Farr. The second set of data may be all the remaining healthcare data for Sarah Fan received from the first source. In this case, the remover 224 may remove all of the non-gynecological data that is not a part of the first set of data and generate the custom documentation template comprising the first set of data. Additionally, based on the one or more indications received by the indication receiver 212, the dynamic bi-directional documentation building system 200 may, via the provider 226, provide only a first portion of the custom documentation template to the gynecologist/clinician (e.g. first user) on the user interface. In aspects, the first portion may be the entire custom documentation template generated comprising the first set of data that satisfied the first criteria (e.g. all data related to an individual available, without limitation to specific medical conditions or limited to a medical specialty such as gynecological health or cardiac health). In other aspects, it is contemplated that only the portion of the template may be provided. The portion provided may include data/fields relevant to the particular encounter taking place, data associated with a predefined period of time (e.g., last 5 years), and the like. Such a presentation of only a portion of the information provides a template that has been customized by the dynamic bi-directional documentation building system 200 to remove information that is not relevant (e.g., it may not be relevant at age 57 to have the data on what type of birth control was taken at age 18 by individual Sarah Farr). Filters to remove information that is not relevant may be customized by users and may be based on, for example, age, sex, current medications, allergies, medical conditions, and the like.

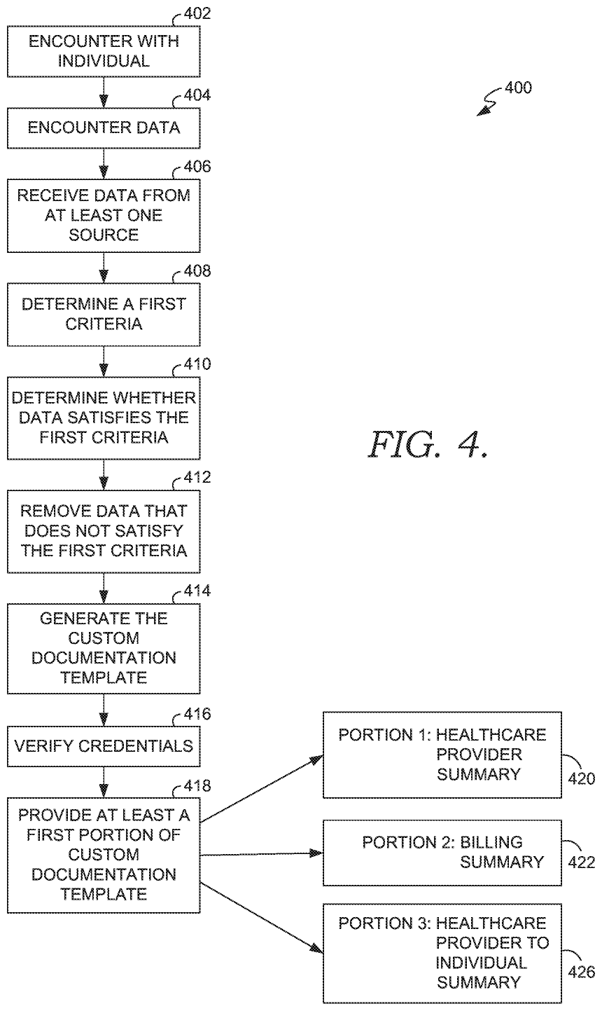

FIG. 4 illustrates an exemplary process illustrating the dynamic bi-directional documentation building system (such as dynamic bi-directional documentation building system 200) generating a custom documentation template and providing customized portions of the custom documentation template to different users (e.g., based on role, etc.). In the aspect shown, the process beings with an individual encounter at block 402. The encounter may be an individual visit with a healthcare provider/clinician or an unplanned event such as a visit to the emergency room. At block 404, encounter data is received, which may include biographical information such as the name, gender, date of birth of the individual, and the like. Additionally, encounter data may include a description regarding why the encounter is taking place (e.g., a chief complaint). For example, the encounter data received at block 404 may indicate that an individual is reporting to the emergency room due to chest pains and shortness of breath. In another instance, the encounter data may indicate that an individual is reporting for a scheduled follow up appointment after surgery (e.g., post-op appointment following knee replacement). Based on the encounter data received at block 404, the dynamic bi-directional documentation building system 200 receives data from at least one source (e.g. the first source) via the data receiver 220 at block 406. As previously mentioned, the at least one source may be another application within the dynamic bi-directional documentation building system 200, such as an electronic heath record application, or may be a database, such as database 204. It is contemplated that the at least one source may be any source from which the data receiver 220 is able to receive data associated with the individual for the building of the custom documentation template.

The criteria determiner 214 determines a first criteria for the custom documentation template at block 408. As mentioned, the first criteria may be narrow or very broad and is customizable to user preferences. If the individual is reporting to the emergency department complaining of various symptoms that may be related to a heart attack, the first criteria determined by the criteria determiner 214 may be broad and include all previous medical history related fields, including past medications and family history, that a user, such as an emergency room physician, may need in order to assess and input as much information regarding the individual as possible for effective treatment. In other circumstances, the first criteria may be more narrow, as described previously for encounters with medical specialists. For example, if an encounter is taking place with a dermatologist, the first criteria may limit the data needed to only the information that is relevant to treating a skin condition. The criteria may be dynamically selected by the dynamic bi-directional documentation building system 200 at the time of the encounter based on programmed logic to assist in the decision making related to criteria. The user may adjust the criteria at any time to expand or further narrow the criteria applied to the template.

Utilizing the criteria, the dynamic bi-directional documentation building system 200 may determine which set of data satisfies the at least one criteria at block 410. Once again, the data included in the first set of data will vary depending on the encounter type and/or the criteria. The first set of data includes data that satisfies the first criteria and the second set of data includes data that does not satisfy the first criteria. After this determination, the remover 224 may remove, at block 412, any data (e.g., the second set of data) that does not satisfy the first criteria. While FIG. 4 illustrates only a first criteria determination, it is contemplated that in other aspects, more than one criteria may be determined and applied to the template based on the data associated with the individual from a first source.

Once the second set of data is removed, the generator 218 may generate the custom documentation template at block 414. Prior to providing the custom documentation template to a user, the dynamic bi-directional documentation building system 200 may verify user credentials at block 416. This is important so that individuals' privacy remains intact and only approved users have access to an individual's EHR in its entirety. After the one or more credentials are verified, the provider 226 provides at least a first portion of the custom documentation to at least one user at block 418. Depending on an identity of the user and their respective role (e.g., billing clerk vs. clinician), the provider 226 may provide different portions of the custom documentation template generated to each user based on their respective roles, as previously described herein.

As shown in FIG. 4, three different portions of the custom documentation template are shown as being provided to a user at blocks 420, 422, and 426. Not all information will be automatically generated for each user. The provider 226 may provide each portion based on the individual user and their specific credentials. The one or more credentials may be verified by the dynamic bi-directional documentation building system 200 prior to providing the at least one portion of the custom documentation template. The one or more credentials utilized to verify the first user or any subsequent users may include, but is not limited to, a user's name, title, and/or a reason for requesting the at least one portion of the custom documentation template. While three different portions of the custom documentation template generated are shown, it is contemplated that there may be any number of portions in certain aspects. In this instance, the provider 226 may provide a first portion, a healthcare provider summary to a healthcare provider at block 420. The provider 226 may provide a second portion, the billing summary, of the custom documentation template to a billing clerk for processing at block 422. The provider 226 may provide a third portion, a healthcare provider to individual summary, to a patient/individual at block 426. As will be discussed further, each portion of the custom documentation template may comprise some overlapping data such as the biographical information for an individual. However, the purpose of contextualizing and customizing the documentation template is so that each different user within a healthcare management system, including an individual being treated, is provided with the most effective custom documentation template for processing and further input as needed.

Providing different portions of the custom documentation template to different users also addresses the issue of information overload that many healthcare providers face when conducting individual examinations. For example, both an internal medicine physician (a.k.a. internist) and an orthopedic surgeon within a healthcare system may be treating the same individual. As such, when the internist has an encounter with the individual, they might be seeing all the orthopedists detailed knee examination notes when the internist selects the physical examination section. As such, the internist is overloaded with information that is not relevant to his general physical examination of the individual. Therefore, providing specific contextualized portions of the customized documentation template based on the user, helps resolve the information overload issue. It also removes the burden from the healthcare provider to either manually remove the excess data and allows each individual healthcare provider to input and visualize the data relevant to their specialty or the encounter situation.

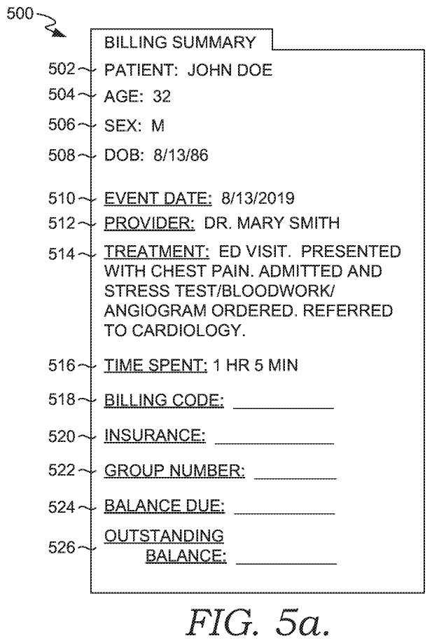

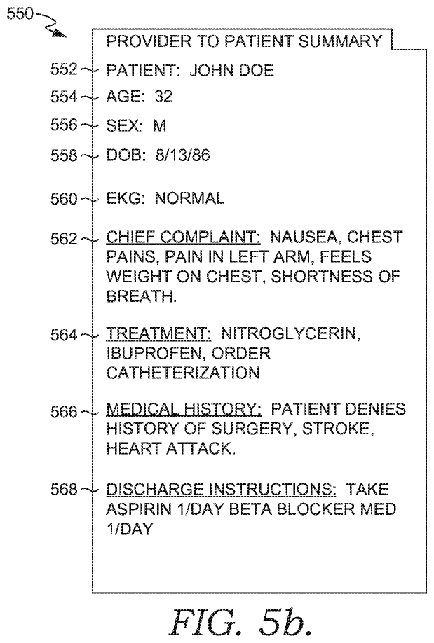

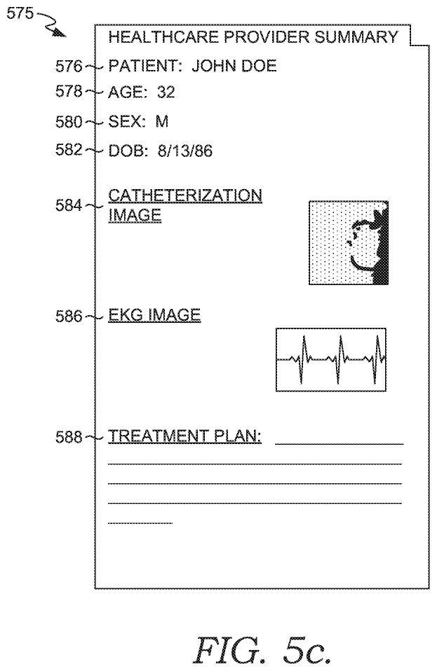

Turning to FIGS. 5a-5c, three different portions of a custom documentation template provided to different users are depicted. FIG. 5a illustrates a billing summary portion 500 of the custom documentation template that may be provided to a billing clerk. As shown, the billing summary documentation template includes the patient name 502, age 504, sex 506, date of birth 508, event date 510, provider 512, treatment 514, time spent with individual 516, billing code 518, insurance 520, group number 522, balance due 524, and outstanding balance 526. The data included in the billing summary portion 500 includes data from the first set of data that satisfied the first criteria previously identified. Continuing with the example regarding an individual seeking care in the emergency room, the billing summary portion 500 of the custom documentation template generated includes information regarding the emergency room encounter. The billing summary portion 500 has data fields 518-526 relating to the billing and insurance for the treatment provided. The generator 218 generated the fields including billing code 518, insurance 520, group number 522, balance due 524, and outstanding balance 526 for input by a billing individual. Additionally, the billing summary includes the basic biographical information, including patient name 502, age 504, sex 506, and date of birth (DOB) 508, the event date 510, the provider 512, the treatment 514, and the treatment provided 514. However, details regarding the individual's medical history, medications, or other confidential health information is not provided in accordance with proper privacy practices in the custom documentation template portion provided for billing. By providing only a specific portion of the custom documentation template relevant to the individual user by removing information not relevant to a user's identified role, the system is improving efficiency and prioritizing maintaining confidentiality of individual health information.

FIG. 5b illustrates a healthcare provider to patient summary 550. This summary includes some of the same biographical information seen in FIG. 5a, including the patient name 552, age, 554, sex 556 and date of birth 558. However, unlike the billing summary portion 500 shown in FIG. 5a, FIG. 5b includes confidential information including EKG results 560, chief complaint 562, treatment 564, medical history 566, and discharge instructions 568. This portion of the custom documentation template is designed to be provided to the individual as a summary upon discharge from the emergency room (or other healthcare facility) and provides a brief summary of the encounter. The provider 226 may provide the specific summary for the individual and include the relevant information that an individual will need for their records.

FIG. 5C illustrates a third portion of the custom documentation template generated, a healthcare provider summary 575 for the healthcare provider. As seen in FIGS. 5a and 5b, the biographical information 576-582 remains the same. However, the healthcare provider summary 575 further includes a catheterization image 584 and EKG image 586, which are provided for review and analysis by the healthcare provider. Additionally, a treatment plan 588 field is provided to receive input from the healthcare provider. This portion of the custom documentation template for individual John Doe provides relevant medical information from a first source associated with John Doe and also allows the healthcare provider to input relevant information for treatment of John Doe. While not shown in FIG. 5C, it is contemplated the healthcare provider summary may also include tables to represent additional clinical information such as lab results and bi-lateral examinations. Additionally, the healthcare provider summary 575 may also be configured to show timeline views demonstrating the timeline of medical issues for an individual and graphical views or trends in the patient's medical history and conditions. Further, by customizing the documentation template to each individual's needs, FIGS. 5a-5c illustrate how the custom documentation template building process can lead to more streamlined, efficient, effective, and privacy-compliant documentation templates. These figures also illustrate how the system contextualizes the portions of a custom documentation template for each user and then provides the appropriate portion of the custom documentation template to each user based on their credentials and needs.

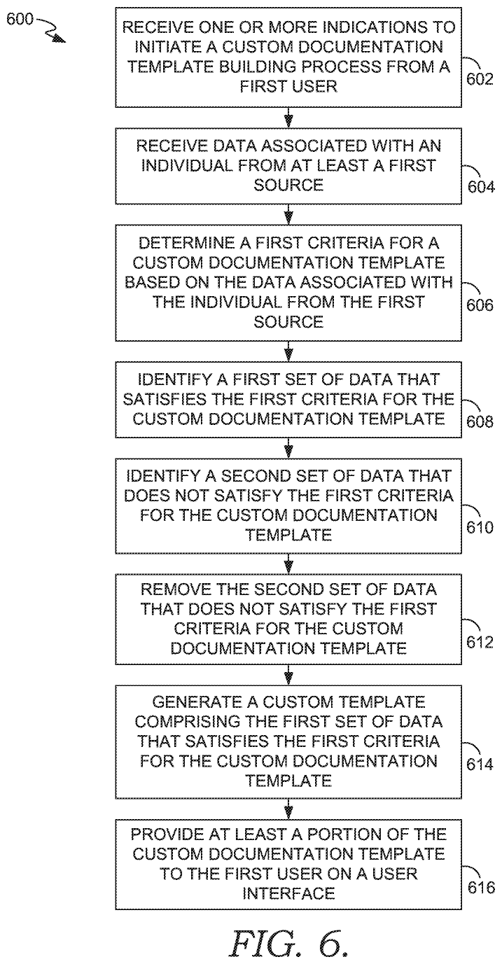

FIG. 6 illustrates an exemplary method 600 for building at least one custom documentation template, in accordance with aspects herein. The method 600 begins at block 602 where one or more indications to initiate a custom documentation template building process from a first user is received by the indication receiver 212. The one or more indications received from the first user may occur during an encounter with an individual, prior to an encounter (e.g., prior to a scheduled appointment with a healthcare provider), or after the encounter for recordkeeping purposes. Additionally, the one or more indications may occur as a result of the individual inputting at least a first set of data for an individual by the first user. For example, the first user may input biographical data such as, but not limited to, the date of birth, sex, gender, and social security number into the dynamic bi-directional documentation building system 200. Biographical data may also include a summary of medical conditions, treatments (past and present), and financial information for an individual. The indication receiver 212 may receive an indication to initiate the custom documentation template building process as a result of receiving the input by the first user.

At block 604, the data receiver 220 receives data associated with an individual from at least a first source. Following this, the criteria determiner 214 determines a first criteria for a custom documentation template based on the data associated with the individual from the first source at block 606. Then, a first set of data that satisfies the first criteria for the custom documentation template is identified by the first data set identifier 222 at block 608. Additionally, a second set of data that does not satisfy the first criteria is identified by the second data set identifier 216 at block 610. Once the second set of data is identified, the remover 224 removes the second set of data that does not satisfy the first criteria at block 612, leaving the first set of data that satisfies the first criteria. The second set of data may be removed from a pool of data such that it is not input into a custom documentation template. Alternatively, a documentation template may be generated and then the second set of data may be removed, thus creating a custom documentation template.

Continuing on with block 614, the generator 218 generates a custom documentation template comprising the first set of data that satisfied the first criteria for the custom documentation template. Once the custom documentation template is generated, the provider 226 provides at least a portion of the custom documentation template to the first user on a user interface at block 616. As previously mentioned, the first user may provide input in the custom documentation template based on an encounter and the updates to the custom documentation template may be saved for future use in, for example, database 204. While not shown, method 600 may further include additional steps, including providing a second portion of the custom documentation template to a second user. The second user may be another healthcare provider, another individual associated with the healthcare management system, or in some instances, may even be the individual.

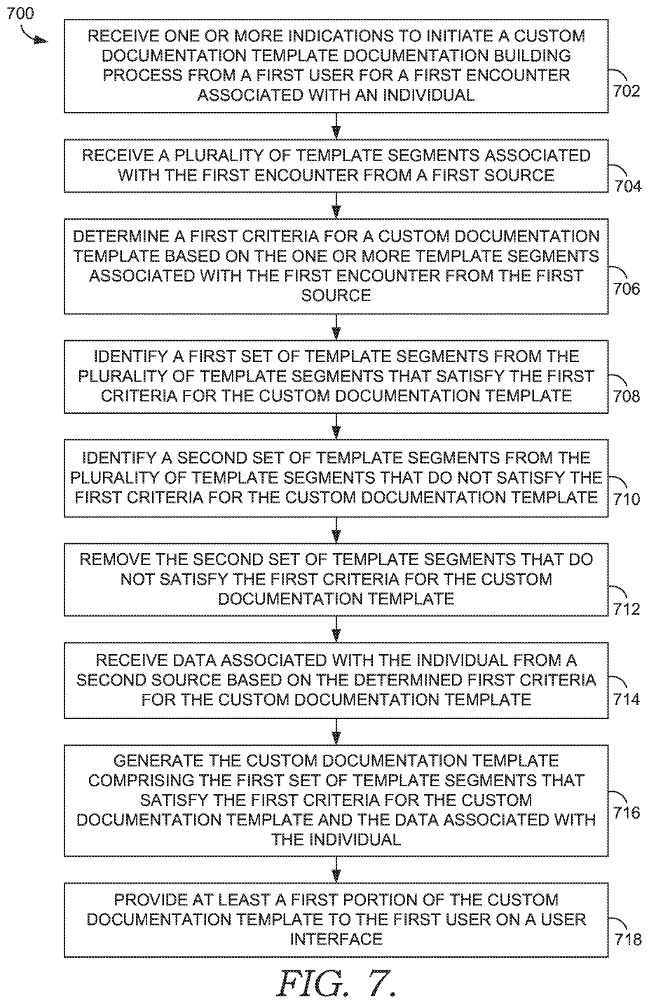

Next, FIG. 7 illustrates another exemplary method 700 for building at least one custom documentation template, in accordance with aspects herein. The method 700 begins with block 702, when one or more processors of a dynamic bi-directional system receives one or more indications to initiate a custom documentation template building process from a first user. In this aspect, the one or more indications to initiate a custom documentation template building process is a result of a first encounter with an individual. As discussed, the first encounter may be a scheduled event (e.g., checkup with primary care physician or appointment with a specialist such as a cardiologist) or an unscheduled event (e.g. emergency room visit).

Next, the dynamic bi-directional documentation building system 200 receives a plurality of template segments associated with the first encounter from a first source at block 704. The plurality of template segments received may include questions, answers, and fields related to the first encounter. For example, if the encounter is a scheduled visit with the cardiologist, the plurality of template segments received may include questions related to the current cardiac health of the individual (e.g., heart rate, followed by an open text field for the cardiologist to record the heart rate at the appointment; cardiac medications followed by a yes/no response field and then an open text field for completing whether any cardiac medications are being taken by the individual). Additionally, the plurality of template segments may include template segments that are not related to the specific medical issue or encounter, such as weight, height, occupation, gender, and other more generalized or biographical information that may be relevant for the customized documentation template to be generated.

Following this, the dynamic bi-directional documentation building system 200 will determine, via the criteria determiner 214, a first criteria for a custom documentation template based on the one or more template segments received from the first source at block 706. The first source may be a database, such as database 204, or any other source that comprises one or more template segments for the generation of a customized documentation template. As discussed, the first criteria for the custom documentation template may be broad or narrow (e.g., all template segments relevant to the medical care of an individual v. all template segments related only to the cardiac care of the individual). Then, a first set of template segments from the plurality of template segments that satisfy the first criteria are identified at block 708. Additionally, a second set of template segments from the plurality of template segments that do not satisfy the first criteria for the custom documentation template are identified at block 710. Once the second set of template segments are identified, the second set of template segments are removed at block 712 by the remover 224. As such, the first set of template segments remains for the generation of the custom documentation template.

Continuing with block 714, the dynamic bi-directional documentation building system 200 will receive data associated with the individual from a second source based on the determined first criteria for the custom documentation template. It is contemplated the second source may be an EHR, database, or any other source from which the dynamic bi-directional documentation building system 200 may receive data associated with the individual. Once the data associated with the individual is received from a second source, the generator 218 will generate the custom documentation template comprising the first set of template segments that satisfy the first criteria for the custom documentation template and the data associated with the individual at block 716. In some aspects, when the custom documentation template is generated with the first set of template segments and data associated with the individual, certain portions of the custom documentation template may already be completed. For example, if a portion of the data received associated with the individual is biographical information (e.g., age, sex, weight, date of birth, social security number, etc.), the system may generate the custom documentation template with this data already completed. Once the custom documentation template is generated, at least a first portion of the custom documentation template is provided, by the provider 226, to the first user on a user interface at block 718. As mentioned, the first user may provide input in the custom documentation template based on an encounter and the updates to the custom documentation template may be saved for future use in, for example, database 204. While not shown, method 700 may further include additional steps, including providing a second portion of the custom documentation template to a second user. The second user may be another healthcare provider, another individual associated with the healthcare management system, or in some instances, may even be the individual.

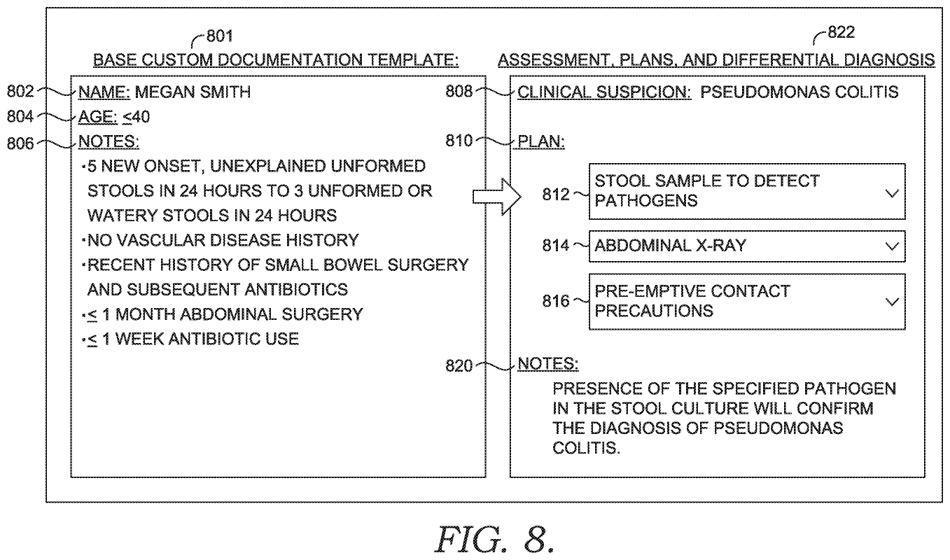

Next, FIG. 8 illustrates another exemplary custom documentation template generated via the custom documentation building process described herein. As mentioned, the custom documentation template may be generated utilizing one or more base custom documentation templates and then, based on input from a user and data received, may be further customized and contextualized for the specific event taking place. For example, the indication receiver 212 receives an indication to initiate a custom documentation template building process from a first user via a user interface to begin the process. A base custom documentation template may be generated and the criteria determiner 214 determines the sections needed for the encounter type and then filters the questions to be included in the custom documentation template from a library of questions so that the only questions included are those that meet the criteria determined by the criteria determiner 214. Data that does not satisfy a first criteria determined is removed by the remover 224 and the generator 218 will generate the base custom documentation template. Once the base custom documentation template is generated by the generator 218 and provided by the provider 226 to the user (e.g. healthcare provider), the user may select or enter values into the template. Then, based on the information entered by the user, additional questions for the documentation template may be dynamically generated based on the selections and context. As shown in FIG. 8, a base custom documentation template 801 is generated for a healthcare provider for determining the “Assessment, Plans, and Differential Diagnosis” for an individual during an office visit. An individual's symptoms, clinical characteristics, age, race, lab values, pending labs, and gender will be factored into the base custom documentation template 801 presented to the healthcare provider for input and used to generate a custom documentation template 822 that includes the data related to the assessment, plan, and differential diagnosis of the individual based on the data generated and input into the base custom documentation template 801.

The base custom documentation template 801 of FIG. 8 includes the patient name 802, age, 804, and a notes 806. In the base custom documentation template 801, encounter data, including the patient name 802 and age 804 is received. Additionally, encounter data may include (not shown) a description regarding why the encounter is taking place (e.g., a chief complaint). Based on the encounter data, the system, such as dynamic bi-directional documentation building system 200, receives data from at least one source (e.g., the first source) via the data receiver 220. As previously mentioned, the at least one source may be another application within the dynamic bi-directional documentation building system 200, such as an electronic heath record application, or may be a database, such as database 204. It is contemplated that the at least one source may be any source from which the data receiver 220 is able to receive data associated with the individual for the building of the custom documentation template. The data received via the data receiver 220 may include data included in the Notes 806 section, such as the data that the individual had abdominal surgery less than a month ago. In some instances, this data may be input directly from the healthcare provider during an encounter or may be received from other sources within the dynamic bi-directional documentation building system 200 (e.g. databases, EHR, etc.).

The criteria determiner 214 determines a first criteria for the custom documentation template. As mentioned, the first criteria may be narrow or very broad and is customizable to user preferences. In FIG. 8, the encounter taking place is between a gastroenterologist and an individual. As such, the first criteria determined by the criteria determiner 214 will be narrow and consists of data related to gastrointestinal care. Utilizing the criteria, the dynamic bi-directional documentation building system 200 may determine which set of data satisfies the at least one criteria. Once again, the data included in the first set of data will vary depending on the encounter type and/or the criteria. The first set of data includes data that satisfies the first criteria and the second set of data includes data that does not satisfy the first criteria. After this determination, the remover 224 may remove any data (e.g., the second set of data) that does not satisfy the first criteria.

Once the second set of data is removed, the generator 218 may generate the custom documentation template 822 for the assessment, planning, and differential diagnosis. As shown, the dynamic bi-directional documentation building system 200 utilizes the data from the base custom documentation template 801 to output the assessment, planning, and differential diagnosis found in custom documentation template 822. The assessment, planning, and differential diagnosis found in custom documentation template 822 includes the clinical suspicion 808 determined based on the analysis of the data in the base custom documentation template 801, the plan 810, and notes 820. In this example, based on the data input and generated in the base custom documentation template 801, the clinical suspicion 808 is determined to be pseudomonas colitis. As such, the user has selected three items as part of the plan 810 to treat the individual from a drop down menu. First, the user has selected to collect and evaluate a stool sample to detect pathogens or toxins at block 812. Second, the user has selected obtaining an abdominal x-ray at block 814. Third, the user has selected beginning pre-emptive contact precautions at block 816. The user has also added notes 820 indicating that the presence of the specific pathogen in the stool culture will confirm the diagnosis of pseudomonas colitis. This process illustrates how template segments are utilized (e.g., patient name, age, notes) and filtered to include the questions related to the encounter (e.g., gastroenterologist visit). Then, based on the data received and input by a user in the base custom documentation template 801, the generator 218 can generate the custom assessment, plan, and differential diagnosis found in custom documentation template 822 that is provided to the user by provider 226 for review and further input.

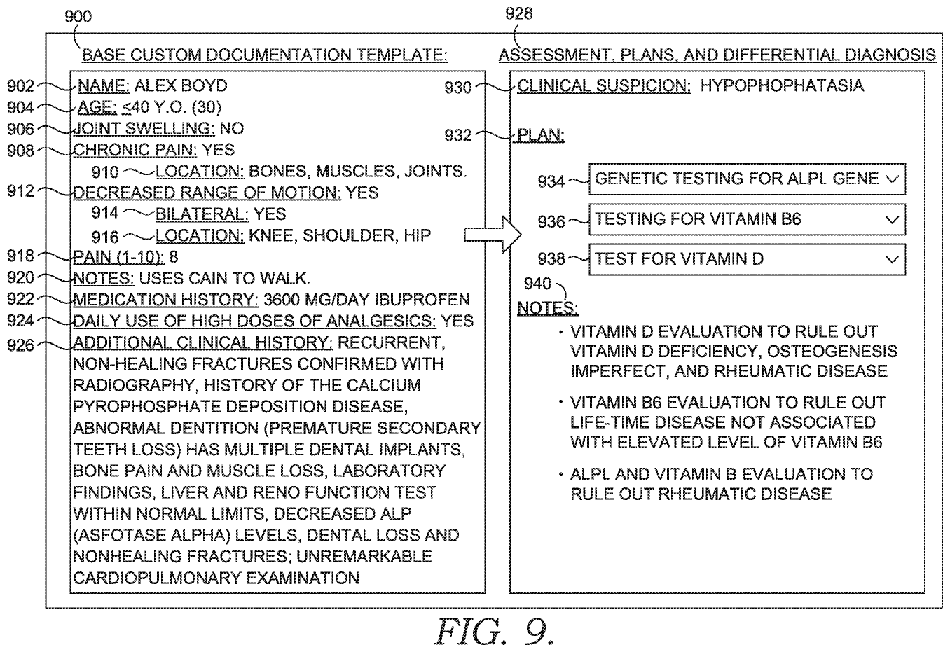

FIG. 9 illustrates yet another custom documentation template 928 for the assessment, plan, and differential diagnosis for the individual generated by the generator 218 based on a base custom documentation template 900. As shown, the dynamic bi-directional documentation building system 200 receives relevant clinical data for an individual for each section of the base custom documentation template 900. In this example, the base custom documentation template includes the name 902, age 904, joint swelling status 906, chronic pain status 908, chronic pain location 910, whether there is decreased range of motion 912, whether the decreased range of motion is bilateral 914, the location of the decreased range of motion 916, pain level 918, notes 920, medication history 922, whether there is daily use of high doses of analgesics 924, and additional clinical history 926. Utilizing the data received in the base custom documentation template, the dynamic bi-directional documentation building system 200 generates, via the generator 218, the custom documentation template 928 which is provided to the provider 226 for review and further input to make the assessment, plan, and differential diagnosis for the individual. As shown, the custom documentation template 928 includes the clinical suspicion indicator 930, the plan indicator 932, and notes indicator 940. The recommendation presented in the custom documentation template 928 includes genetic testing for the ALPL gene at indicator 934, testing for Vitamin B6 at indicator 936, and testing for Vitamin D at indicator 938. Additionally, the user and/or dynamic bi-directional documentation building system 200 has generated notes at notes indicator 940 corresponding to the recommendation at plan indicator 932. As seen in FIGS. 8 and 9, the base custom documentation templates 801 and 900 are further customized and contextualized based on data received and user input to generate a customized documentation template to aid the healthcare provider in assessing the individual's condition, and determining a treatment plan and differential diagnosis.

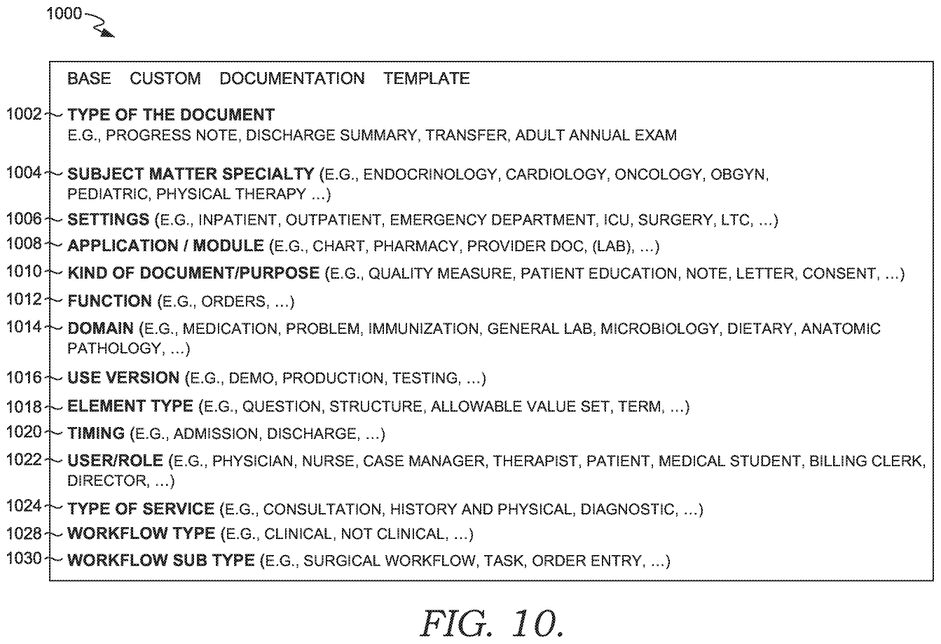

FIG. 10 illustrates exemplary template segments for an exemplary base custom documentation template to be generated by the generator 218. As shown, the base custom documentation template 1000 includes several template segments 1002-1030 that are utilized to generate a custom documentation template such as custom documentation template 928 of FIG. 9. The first template segment is the type of document 1002 which includes selection options for the type of base documentation template to be generated, such as a progress note, discharge summary, or annual exam. A user or the dynamic bi-directional documentation building system 200 can determine the appropriate selection based on input and/or data received. Next, the base custom documentation template 1000 may be customized based on the subject matter specialty 1004 (e.g., cardiology, oncology, etc.). Further, the base custom documentation template 1000 may be customized based on the settings 1006, which include whether the encounter is an inpatient encounter or an outpatient encounter and provide department details. Several additional template segments to be determined and included in the base custom documentation template 1000 include: the application/module 1008 (whether the application is in the individual's chart, pharmacy, or elsewhere), the kind of documentation 1010 (patient education, letter, consent, etc.), the function 1012 (orders), the domain 1014 (medication, immunization, labs, etc.), the use version 1016 (demo, testing, production, etc.), the element type 1018 (whether the template segment is a question, value set, term, etc.), the timing of the encounter 1020 (admission, discharge), user/role 1022 (physician, nurse, billing clerk, etc.), type of service 1024 (consultation, physical, diagnostic, etc.), the workflow type 1028 (clinical, not clinical) and workflow sub type 1030 (surgical, task, order entry, etc.). Based on the data received by the data receiver 220, criteria determined by the criteria determiner 214, the first set of data and second set of data identified by the first data set identifier 222 and the second data set identifier 216, and the removal of the data not meeting the criteria determined by the remover 224, a customized documentation template will be generated by the generator 218 utilizing the template segments 1004-1030 shown in FIG. 10. Based on the selection of the template segments 1004-1030, different base custom documentation templates 1000 may be generated for different users, allowing a variety of input and contextualization based on each specific user's needs.

The present invention has been described in relation to particular embodiments, which are intended in all respects to be illustrative rather than restrictive. Further, the present invention is not limited to these embodiments, but variations and modifications may be made without departing from the scope of the present invention.

What is claimed is:

1. A dynamic bi-directional documentation building system for building at least one custom documentation template, the system comprising one or more processors configured to:
receive one or more indications to initiate a custom documentation template building process from a first user;
receive data associated with an individual from at least a first source;
determine a first criteria for a custom documentation template based on the data associated with the individual from the first source;
identify a first set of data that satisfies the first criteria for the custom documentation template;
identify a second set of data that does not satisfy the first criteria for the custom documentation template;
remove the second set of data that does not satisfy the first criteria for the custom documentation template;
generate a custom documentation template comprising the first set of data that satisfies the first criteria for the custom documentation template; and
provide at least a first portion of the custom documentation template to the first user on a user interface.

2. The system of claim 1, wherein the one or more indications to initiate a custom documentation template building process occurs as a result of receiving input of at least one piece of data for an individual by the first user during an encounter with the individual.

3. The system of claim 2, wherein the input of at least one piece of data for the individual comprises input of biographical data.

4. The system of claim 3, where in the biographical data for the individual comprises at least one of a summary of medical conditions, treatment, and financial information.

5. The system of claim 1, wherein the first source is an electronic health record.

6. The system of claim 1, wherein the one or more processors are further configured to receive data for the custom documentation template from a second source.

7. The system of claim 6, wherein the second source is a database comprising one or more custom documentation templates previously generated.

8. The system of claim 1, wherein the one or more processors are further configured to verify one or more credentials of the first user prior to providing the at least first portion of the custom documentation template to the first user.

9. The system of claim 8, wherein the one or more credentials for the first user includes at least one of a name of a user, a user's title, or at least one reason for requesting the custom documentation template.

10. The system of claim 1, wherein the custom documentation template is stored for future use.

11. The system of claim 1, wherein at least a second portion of the custom documentation template is provided to a second user on a user interface.

12. The system of claim 11, wherein the second portion of the custom documentation template provided to the second user is different from the first portion of the custom documentation template provided to the first user.

13. The system of claim 1, wherein the system further receives a set of rules and data fields to be included in the custom documentation template from a second source.

14. A method for building at least one custom documentation template by a dynamic bi-directional documentation building system, the method comprising:
receiving one or more indications to initiate a custom documentation template building process from a first user;
receiving data associated with an individual from at least a first source;
determining a first criteria for a custom documentation template based on the data associated with the individual from the first source;
identifying a first set of data that satisfies the first criteria for the custom documentation template;
identifying a second set of data that does not satisfy the first criteria for the custom documentation template;
removing the second set of data that does not satisfy the first criteria for the custom documentation template;
generating a custom documentation template comprising the first set of data that satisfies the first criteria for the custom documentation template; and
providing at least a first portion of the custom documentation template to the first user on a user interface.

15. The method of claim 14, wherein the one or more indications to initiate a custom documentation template building process are received during an encounter with the individual.

16. The method of claim 15, further comprising receiving annotations to the portion of the custom documentation template based on the encounter with the individual.

17. The method of claim 14, wherein a second portion of the custom documentation template is provided to a second user.

18. The method of claim 17, wherein the second portion of the custom documentation template provided to the second user is different from the first portion of the custom documentation template provided to the first user.

19. A dynamic bi-directional documentation building system for building at least one custom documentation template, the system comprising one or more processors configured to:
receive one or more indications to initiate a custom documentation template documentation building process from a first user for a first encounter associated with an individual;
receive a plurality of template segments associated with the first encounter from a first source;
determine a first criteria for a custom documentation template based on the one or more template segments associated with the first encounter from the first source;
identify a first set of template segments from the plurality of template segments that satisfy the first criteria for the custom documentation template;
identify a second set of template segments from the plurality of template segments that do not satisfy the first criteria for the custom documentation template;
remove the second set of template segments that do not satisfy the first criteria for the custom documentation template;
receive data associated with the individual from a second source based on the determined first criteria for the custom documentation template;
generate the custom documentation template comprising the first set of template segments that satisfy the first criteria for the custom documentation template and the data associated with the individual; and
provide at least a first portion of the custom documentation template to the first user on a user interface.

20. The system of claim 19, wherein the first source is an application comprising a set of rules and data fields to be included in the custom documentation template.
